(19) United States
(12) Patent Application Publication
(10) Pub. No.: US 2025/0073272 A1
(43) Pub. Date: Mar. 6, 2025

(54) PRODUCTION OF ENHANCED STEM CELL-BASED EXOSOMES AND USES IN SCAR TISSUE PREVENTION AND TREATMENT

(71) Applicants: Sharan D. RAMASWAMY, Miami, FL (US); Manuel Perez NEVAREZ, Miami, FL (US); Asad MIRZA, Miami, FL (US); Yih-Mei LIN, Miami, FL (US); Amanda IBARRA, Miami, FL (US); Pranavi ARMAN, Miami, FL (US)

(72) Inventors: Sharan D. RAMASWAMY, Miami, FL (US); Manuel Perez NEVAREZ, Miami, FL (US); Asad MIRZA, Miami, FL (US); Yih-Mei LIN, Miami, FL (US); Amanda IBARRA, Miami, FL (US); Pranavi ARMAN, Miami, FL (US)

(73) Assignee: THE FLORIDA INTERNATIONAL UNIVERSITY BOARD OF TRUSTEES, Miami, FL (US)

(21) Appl. No.: 18/240,681

(22) Filed: Aug. 31, 2023

(51) Int. Cl. A61K 35/28; A61K 9/00; A61K 9/127; A61K 38/17; A61K 38/18; A61K 38/19; A61K 38/20; A61K 38/22; A61K 38/30; A61K 38/38; A61K 38/40; A61P 9/10; C12N 5/0775

(52) U.S. Cl. CPC A61K 35/28; A61K 9/0019; A61K 9/127; A61K 38/1722; A61K 38/1808; A61K 38/1825; A61K 38/1841; A61K 38/1858; A61K 38/1866; A61K 38/191; A61K 38/195; A61K 38/2006; A61K 38/204; A61K 38/22; A61K 38/2264; A61K 38/30; A61K 38/385; A61K 38/40; A61P 9/10; C12N 5/0663; C12N 2501/115; C12N 2521/00

(57) ABSTRACT

This disclosure pertains to a non-living biological product. Particularly, exosomes derived from stem cells can help prevent or reduce scar tissue growth in cardiovascular system and restore heart function. According to certain embodiments, a fluid-induced, pathological shear stress mechanical stimulation process of stem cells is used to augmented quantity and quality of exosomes produced from stem cells. These exosomes serve as a therapeutic agent for preventing or reducing scar tissue growth in cardiovascular system. Therefore, compositions comprising the exosomes derived from stem cells and methods of preventing and/or treating scar tissue growth in cardiovascular system by administering the exosomes isolated from stem cells are also provided.

### GOVERNMENT SUPPORT

This invention was made with government support under 1940141 awarded by National Science Foundation. The government has certain rights in the invention.

### BACKGROUND

Heart disease is the leading cause of death for both men and women in the United States. Coronary heart disease is the most common type of heart disease. Every year approximately 790,000 Americans have a heart attack, resulting in over 370,000 deaths per year.

In coronary artery disease, the arteries that supply blood to the heart become occluded due to plaque build-up in the arterial walls. If the obstruction is severe enough, it can prevent blood from reaching portions of the heart muscle, resulting in myocardial infarction (MI), leading to irreversible damage and necrosis of the heart tissue. The resulting cardiomyopathy produces scar tissue in the affected muscles, inhibiting the heart's ability to properly pump blood, which in turn can lead to heart failure.

Cardiac scar tissue is formed after the ventricular wall of the heart necroses due to damage. In contrast to myocardial tissue, cardiac scar tissue contains no cardiac muscle cells. Instead, it is composed of connective tissue cells, such as fibroblasts, and non-cellular components, such as collagen and fibronectin. Cardiac scar tissue is non-contractile, and, therefore, interferes with normal cardiac function.

Although current medical devices such as stents and vascular grafts provide effective management to a sub-set of patients, they have distinct limitations and, importantly, do not restore the health of cardiac tissues that have already been damaged by a heart attack.

Current treatments for ischemic cardiomyopathy focus on minimizing the existing post-infarct damage to the myocardium and avoiding further progression of fibrotic lesions. Nonetheless, the initial lesion persists, and the subsequent formation of non-contractile fibrotic tissue leads to infarct expansion and extension, which reduces the functional capacity of the heart and can lead to heart failure. The use of stem cells as a therapeutic strategy for the treatment of cardiac diseases has emerged in the last two decades. The potential for treating ischemic cardiomyopathy via neovascularization of the heart thereby leading to regeneration of myocardial tissues has been the driving force of these stem cell investigations.

Recently, stem cell-injection therapy has been investigated as an option to treat the infarcted myocardium with the goal of regenerating cardiac muscle and restoring heart function. Over 244 clinical trials have been initiated to explore the use of stem cells to treat ischemic cardiomyopathy. Mesenchymal stem cells (MSCs) such as bone marrow mesenchymal stem cells (BMSCs) are considered to hold great therapeutic value for cell-based therapies and tissue regeneration. Numerous preclinical studies of ischemic cardiomyopathy employing an MSC-based therapy have demonstrated that fibrosis, i.e., scar tissue reduction and stimulating cardiomyogenesis, can lead to improvements in the structure and function of remodeled ventricles. Scientists have used the implantation of human, adult BMSCs and human adult cardiac stem cells (CSCs) to induce remodeling and regeneration of affected tissue. However, the use of undifferentiated mesenchymal stem cells has produced only marginal results due to their poor retention and engraftment in the diseased tissue.

Allogeneic BMSCs that were intravenously delivered in baboons (2 million cells/kg-body-weight) immediately following induced MI exhibited neovascularization and capillarization within the cardiac tissues, post-treatment. While these beneficial effects have been reported, the underlying mechanisms are not well understood. It has been observed that after injection into the heart, exogenous MSCs show poor survival and engrafiment into the infarct site, with evidence that MSCs do not persist and disappear 4 weeks after transplantation. This suggests that instead of direct differentiation of MSCs, other secondary or trophic effects are involved in producing the observed benefits.

CSCs have recently showed more favorable results in treating ischemic cardiac diseases. These cells are isolated via biopsies of the heart muscle but are present in the tissue in very low numbers. In addition, being adult stem cells, the expansion potential for these cells is very limited. The result of these barriers is that CSCs are very costly and in limited supply. This is a major pain point in the stem cell market in that the best performing cells for treatment of ischemia-induced cardiac tissue damage are basically unavailable for use in commercial scale applications.

Thus, there is a need for developing products that replicate the benefits of adult CSCs and MSCs while being available in sufficient quantities and at reasonable costs of production, for use as a therapeutic that prevents, inhibits, or slows down scar tissue growth in the cardiovascular system and restores heart function.

### BRIEF DESCRIPTION OF THE INVENTION

The subject invention provides methods for producing stem cell-derived exosomes under pulsatile fluid-induced oscillatory shear stresses. Stem cells cultured under mechanical stimulations produce and subsequently release injectable enhanced stem cell exosomes (IESCEs) into the culture media in a relatively high quality and concentration.

In one embodiment, the subject invention provides a method for producing secreted exosomes of stem cells, the method comprising culturing the stem cells in a bioreactor comprising a pulsatile flow pump that delivers dynamic flow conditions to the cultured stem cells, and isolating the secreted exosomes. Pulsatile flow stem cell culture with increased disturbed flow or flow oscillations that coincides with sites of cardiovascular disease formation enhance stem cell-derived exosomes due to the initial response to counteract and reduce the adverse effects of the pathological flow environment.

In one embodiment, the bioreactor comprises the pulsatile flow pump that delivers an increased or high oscillatory flow condition compared to a physiological oscillatory flow condition. Such increased or high oscillatory flow condition provides a pathological mechanical environment for cultured stem cells. Preferably, the pathologically high oscillatory flow condition has a shear index/oscillatory shear index (OSI) ranging from about 0.3 to about 0.5. In a specific embodiment, the pathologically high oscillatory flow condition has a shear index/OSI of about 0.5.

In one embodiment, culturing the stem cells in the bioreactor comprises seeding the stem cells on a bio-scaffold, placing the bio-scaffold in the bioreactor, and culturing the stem cells under a static and/or dynamic flow condition, e.g., a pathologically high oscillatory flow condition. In a further embodiment, the bio-scaffold comprises PSIS or PGA-PLLA.

In one embodiment, the subject invention provides the injectable enhanced stem cell exosomes (IESCE) secreted from stem cells under a pathologically high oscillatory flow condition. In certain embodiments, the secreted exosomes or IESCE comprise one or more cytokines selected from, for example, tumor necrosis factor alpha (TNF-α), insulin-like growth factor-1 (IGF-1), vascular endothelial growth factor (VEGF), interleukin-6 (IL-6), acidic fibroblast growth factor (FGF-1), transforming growth factor type beta (TGF-β), epidermal growth factor (EGF), leptin, interleukin-α (IL-1α), platelet-derived growth factor (PDGF)-BB, resistin, monocyte chemoattractant protein-1 (MCP-1), and adiponectin.

In specific embodiments, the method for producing the secreted exosomes further comprises detecting the level of one or more cytokines selected from, for example, TNF-α, IGF-1, VEGF, IL-6, FGF-1, TGF-β, EGF, leptin, IL-1α, PDGF-BB, resistin, MCP-1, and adiponectin. In a specific embodiment, IESCE secreted from stem cells under a pathologically high oscillatory flow condition comprises increase levels of the one or more cytokines selected from, for example, TNF-α, IGF-1, VEGF, IL-6, FGF-1, TGF-β, EGF, leptin, IL-1α, PDGF-BB, resistin, MCP-1, and adiponectin.

The subject invention also provides a composition comprising secreted exosomes of BMSCs or CSCs for use as a therapeutic agent for the regenerative repair of diseased heart tissues and for the prevention and inhibition of scar tissue growth in cardiovascular system.

Advantageously, because exosomes are non-living, the need to prolong stem cell survival in the heart is not necessary. These exosomes comprise a cocktail of molecular factors that promote cardiac function via neovascularization and de novo tissue formation. In a preferred embodiment, the secreted exosomes of stem cells comprise one or more cardiac tissue-specific proteins selected from serum albumin, serotransferrin, and alpha-2-macroglobulin.

In a specific embodiment, the composition of the subject invention comprises IESCEs. In one embodiment, the composition and/or secreted exosomes of the subject invention comprise an increased amount of total proteins compared to that from a static culture.

In a preferred embodiment, exosomes or IESCEs are derived i) from statically cultured, adult, human BMSCs; ii) from physiologically-relevant, oscillatory flow conditioned adult, human BMSCs; iii) from pathologically high oscillatory conditioned adult, human BMSCs; iv) from statically cultured, adult, human CSCs; and/or v) from pathologically high oscillatory conditioned adult, human CSCs.

Further embodiments of the invention also provide methods of treating a cardiovascular disease (e.g., myocardial infarction), by administering, to a subject, the enhanced exosomes or a composition comprising secreted exosomes obtained from stem cells cultured under, for example, pathologically high oscillatory conditions.

In one embodiment, the stem cells can be MSCs or CSCs. In preferred embodiments, the MSCs are BMSCs and the CSCs are cardiosphere-derived cells (CDCs), Sca-1+ CSCs, cardiac mesoangioblasts, cardiac side population cells, Islet-1+ CSCs, epicardium-derived progenitor cells, cardiac colony-forming-unit fibroblasts, or W8B2+ CSCs.

In one embodiment, the subject invention also provides a method for promoting cardiac tissue regeneration, the method comprising identifying a damaged cardiovascular tissue in a subject, and administering to the subject the composition or secreted exosomes of the subject invention. In preferred embodiments, IESCE accelerate healing and remodeling responses of cardiovascular structure (e.g., heart, blood vessels, or heart valve) damaged from disease.

In one embodiment, the subject invention provides a method for preventing, inhibiting, reducing, and/or slowing down scar tissue growth in a cardiovascular system, the method comprising administering to a subject in need thereof the composition or secreted exosomes of the subject invention. In a preferred embodiment, the subject has been diagnosed with a cardiovascular disease. In a specific embodiment, the cardiovascular disease is myocardial infarction or cardiomyopathy.

In certain embodiments, the administration is via a local, oral, nasal, topical, transdermal, intravenous, intraarterial, intraventricular, intradermal, subcutaneous or intramuscular route. In a preferred embodiment, the administration is via intra-cardiac injection.

### BRIEF DESCRIPTION OF DRAWINGS

FIGS. 1A-1B. Developed in-house “torpedo” bioreactor. (1A) a design of the bioreactor with the major parts, including the torpedo, and tube inlet and outlet. The torpedo bioreactor that allows connection to a pulsatile flow pump that delivers fluid oscillations that lead to secreted BMSC-derived exosomes in the conditioned media. (1B) Bioreactor schematic filled with cell culture media, the torpedo, and a bio-scaffold on the torpedo with seeded human stem cells (dots). The bioscaffold on the torpedo with seeded BMSCs was subjected to maximum flow oscillations or disturbances (OSI=0.5), which is prevalent in areas of cardiovascular disease.

FIG. 2. Substantial increase in 8 cytokines within exosomes secreted by BMSCs after exposure to a pathologically high flow oscillations (OSI=0.5) compared to a static condition and a physiological flow condition (OSI=0.2).

FIGS. 3A-3B. ELISA colorimetric assay shows the levels of cytokines including VEGF, PDGF-BB, leptin, resistin, IGF-1, IL-1a, MCP-1, and adiponectin in the isolated exosomes after BMSCs were exposed to a pathologically high OSI of 0.5, a physiologically relevant OSI of 0.2 and a static control, respectively. (3A) cytokine levels were compared in static, 0.2 OSI, and 0.5 OSI treatment groups. (3B) ELISA assay results under static, 0.2 OSI, and 0.5 OSI, respectively, were compared between cytokines.

### DETAILED DESCRIPTION

The subject invention provides products and compositions for use as therapeutic agents for preventing, inhibition or slowing down scar tissue growth in cardiovascular system, restoring heart function, regenerating heart tissue, and/or treating cardiovascular diseases. Advantageously, the products and compositions of the subject invention can replicate the benefits of adult CSCs.

The subject invention also provides methods of producing the products and compositions. The subject invention further provides methods of using the compositions and products for preventing, inhibition or slowing down scar tissue growth in a cardiovascular system, restoring heart function, regenerating heart tissue, and/or treating cardiovascular diseases.

### Injectable Enhanced Stem Cell Exosomes (IESCE).

Cells release extracellular vesicles (EVs) into their environment. EVs can be grouped based on their sizes. Exosomes are nanometer-sized membrane-bound vesicles (e.g., 40-150 nm) that are released from most cells when the multivesicular body (MVB) fuses with the plasma membrane. Exosomes differ from other EVs by their biogenesis. The surface of exosomes is composed of mostly cholesterols and phospholipids with proteins and glycans on the surface. Inside exosomes are biomolecules such as proteins, RNA, DNA, and lipids. The composition of exosomes tends to resemble the cells that release them.

For example, exosomes contain cytokines and growth factors, lipids, mRNAs, and regulatory miRNAs. Exosomes also contain a conserved set of proteins including tetraspanins (e.g., CD81, CD63, and CD9), heat-shock proteins (e.g., HSP60, HSP70 and HSP90), targeting or adhesion markers such as integrins, ICAM-1, EpCAM, membrane fusion markers such as annexins, tumor susceptibility gene 101 (TSG101), and ALIX; and may further contain unique tissue-specific proteins that reflect their cellular origins.

Exosomes have many physiological functions such as the transportation of biomolecules, cellular communication, and cellular defense. Exosome functions can also be intertwined with pathological processes. For example, cancer cells release abundant exosomes to deactivate the immune cells, induce angiogenesis for cancer progression, and create a niche environment for cancer cells to migrate into during metastasis.

Exosomes are associated with immune responses, viral pathogenicity, pregnancy, cardiovascular diseases, central nervous system-related diseases, substance use disorders, and cancer progression. Exosomes possess many capabilities from diagnostic to therapeutic applications. Pertaining to oncology, cancer-derived exosomes contain many cancer biomarkers, which is helpful for noninvasive liquor biopsy. Exosomes have also gained attention because of their non-immunogenic properties and high drug-loading ability.

In one embodiment, the exosomes and IESCEs are released from stem cells such as bone marrow stem cells (BMSCs) and CSCs. Preferably, the BMSCs are bone marrow mesenchymal stem cells (MSCs).

The term “mesenchymal stem cell” or “MSC”, as used herein, refers to a multipotent somatic stem cell derived from mesoderm, having self-regenerating and differentiating capacity to produce progeny cells with a large phenotypic variety, including connective tissues, stroma of bone marrow, adipocytes, dermis and muscle, among others. MSCs may be isolated from any type of tissue. Generally, MSCs may be isolated from bone marrow, adipose tissue, umbilical cord, or peripheral blood. In a particular embodiment, the MSC are bone marrow-derived stem cells.

“Bone marrow mesenchymal stem cells,” or “bone marrow derived mesenchymal stem cells” refer to cells existing in the bone marrow, which can be directly collected from the bone marrow or indirectly collected from other tissues (e.g., blood, skin, fat, and other tissues), and can be cultured and proliferated as adherent cells on a solid surface, e.g., a culture dish (made of plastic or glass). These cells are characterized in having a potential to differentiate into mesenchymal tissues such as bone, cartilage, and fat, or into skeletal muscle, heart muscle, nervous tissues, and epithelial tissues, and can be obtained by collection of bone marrow cells. Markers for human bone marrow mesenchymal stern cells can be, for example, all or some of the following, but are not limited to, Lin-negative, CD45-negative, CD44-positive, CD90-positive, and CD29-positive.

The principal mode of action of MSCs is the localized production and secretion of bioactive trophic factors. The predominant method by which stem cells facilitate healing of cardiovascular tissues is via the release of trophic factors that lead to paracrine-mediated events thereby inducing regeneration. Therefore, stem cells are not required to promote repair; rather the factors that they release in a physiologically-relevant and mechanically active environment are solely needed.

The subject invention identifies specific pathologically high oscillatory conditions that can be created using bioreactors to produce IESCE. IESCE can be delivered without a living cellular component, thus reducing concerns associated with contamination risk and hostile immune responses. Also, storage and transportation of liquid media containing potent healing factors, such as the IESCE-product, for optimal delivery in a clinical setting is more straightforward and inexpensive compared to directly using the cells themselves, which would thereby facilitate the manufacturing at-scale of this therapeutic.

Advantageously, IESCE, a non-living biologic, thus, can leverage the beneficial effects of stem cells, e.g., BMSC injections into the damaged heart muscle in a manner that is not cell survival-dependent, thereby enabling restoration of heart function, and preventing and inhibiting scar tissue growth. Additionally, IESCE can be produced in unlimited quantities to improve cardiac function, thereby overcoming supply limitations of CSCs. The IESCE, which is comprised of nonliving trophic factors (e.g., growth factors, cytokines, mi-RNA, and chemokines), can be used to induce cardiac tissue regeneration and prevent and/or inhibit scar tissue growth in a cardiovascular system.

In certain embodiments, IESCE comprises one or more cytokines that include, but are not limited to, tumor necrosis factor alpha (TNF-α), insulin-like growth factor-1 (IGF-1), vascular endothelial growth factor (VEGF), interleukin-6 (IL-6), acidic fibroblast growth factor (FGF-1), transforming growth factor type beta (TGF-β), epidermal Growth Factor (EGF), leptin, interleukin-α (IL-1α), platelet-derived growth factor (PDGF)-BB, resistin, monocyte chemoattractant protein-1 (MCP-1), and adiponectin.

In some embodiments, IESCE comprises tissue repair factors including, for example, VEGF, IL-6, IL-10, TGF-β1, fibroblast growth factor 2 (FGF-2), extracellular matrix metalloproteinase inducer (EMMPRIN), MMP-9, hepatocyte growth factor (HGF) and granulocyte colony stimulating factor (G-CSF).

In certain embodiments, IESCE can be derived from: (i) statically cultured, adult, human BMSCs (ii) physiologically-relevant, oscillatory flow-conditioned adult, human BMSCs; (iii) pathologically high oscillatory conditioned adult, human BMSCs; (iv) pathologically high oscillatory conditioned adult, human CSCs; or (v) statically cultured, adult, human CSCs which have shown to release potent factors beneficial for cardiac regeneration. Note that the IESCE is a non-living biological product and hence lends itself to repeatability in manufacturing at a large-scale, as an off-the-shelf therapeutic.

In one embodiment, the secreted exosomes under a pathologically high oscillatory flow condition of the subject invention comprise a significantly increased amount of total proteins compared to that from a control condition and/or a physiological oscillatory flow condition. The increase in total protein quantity in secreted exosomes according to the subject invention can be about a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, 600%, 700%, 800%, 900%, 1000%, 2000%, 3000%, 4000%, 5000% or more, or any amount of increase in between.

In one embodiment, the exosomes, i.e., IESCE, secreted from stem cells comprises a total protein of at least 1.5 ng/cell, at least 2 ng/cell, at least 2.5 ng/cell, at least 3 ng/cell, at least 3.5 ng/cell, at least 4 ng/cell, at least 4.5 ng/cell, at least 5 ng/cell, at least 5.5 ng/cell, at least 6 ng/cell, at least 6.5 ng/cell, at least 7 ng/cell, at least 7.5 ng/cell, at least 8 ng/cell, at least 8.5 ng/cell, at least 9 ng/cell, at least 9.5 ng/cell, at least 10 ng/cell, at least 11 ng/cell, at least 12 ng/cell, at least 13 ng/cell, at least 14 ng/cell, at least 15 ng/cell, at least 16 ng/cell, at least 17 ng/cell, at least 18 ng/cell, at least 19 ng/cell, at least 20 ng/cell, at least 21 ng/cell, at least 22 ng/cell, at least 23 ng/cell, at least 24 ng/cell, at least 25 ng/cell, at least 26 ng/cell, at least 27 ng/cell, at least 28 ng/cell, at least 29 ng/cell, at least 30 ng/cell, at least 35 ng/cell, at least ng/cell, at least 45 ng/cell, at least 50 ng/cell, at least 55 ng/cell, at least 60 ng/cell, at least 65 ng/cell, at least 70 ng/cell, at least 75 ng/cell, at least 80 ng/cell, at least 85 ng/cell, at least 90 ng/cell, at least 95 ng/cell, at least 100 ng/cell, or any amount in between.

In a specific embodiment, the secreted exosomes comprise a significantly reduced amount of fibronectin compared to that from a control condition. The reduction in fibronectin quantity in secreted exosomes and/or the composition according to the subject invention can be about a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 100%, or any amount of reduction in between.

Fibronectin is an important component of extracellular matrix and blood plasma and it is also a ligand for integrin adhesion receptor. Fibronectin has various functions including embryonic development, wound healing, fibrosis, and immune and inflammatory responses. It is increased during postnatal development and decreased with progression to adulthood. Fibronectin is considered a good source in order to help form a cell supporting matrix for connective tissues and tissue repair after the injury. However, fibronectin is overexpressed after heart injury such as myocardial infarction and atherosclerosis. The overexpressed fibronectin may further contribute to cardiac fibrosis.

In certain embodiments, the secreted exosomes under a pathologically high oscillatory flow condition of the subject invention comprise a significantly increased amount of TNF-α compared to that from a control condition and/or a physiological oscillatory flow condition. The increase in TNF-α quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of IGF-1 compared to that from a control condition and/or a physiological oscillatory flow condition. The increase in IGF-1 quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of VEGF compared to that from a control condition and/or a physiological flow condition. The increase in VEGF quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of IL-6 compared to that from a control condition and/or a physiological flow condition. The increase in IL-6 quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of FGF-1 compared to that from a control condition and/or a physiological flow condition. The increase in FGF-1 quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of TGF-β compared to that from a control condition and/or a physiological flow condition. The increase in TGF-β quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of EGF compared to that from a control condition and/or a physiological flow condition. The increase in EGF quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of leptin compared to that from a control condition and/or a physiological flow condition. The increase in leptin quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of IL-1α compared to that from a control condition and/or a physiological flow condition. The increase in IL-1α quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of PDGF-BB compared to that from a control condition and/or a physiological flow condition. The increase in PDGF-BB quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of resistin compared to that from a control condition and/or a physiological flow condition. The increase in resistin quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of MCP-1 compared to that from a control condition and/or a physiological flow condition. The increase in MCP-1 quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In certain embodiments, the secreted exosomes under a pathologically high oscillatory flow condition of the subject invention comprise a significantly increased amount of adiponectin compared to that from a control condition and/or a physiological flow condition. The increase in adiponectin quantity in secreted exosomes according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

### Method of Producing IESCE

In one embodiment, the subject invention provides a method of producing enhanced exosomes, i.e., IESCE, under a pathological/high oscillatory flow condition. Surprisingly and advantageously, such enhanced exosomes comprise increased levels of cytokines compared to those produced under a static and/or a physiological oscillatory flow condition.

In one embodiment, the subject invention pertains to a bioreactor that, via pulsatile fluid-induced oscillatory shear stresses, can mechanically stimulate stem cells to produce and subsequently release into the culture media exosomes in relatively high quality and concentration. Certain examples of bioreactors that could be used in the methods used in this disclosure are described in U.S. Pat. No. 8,852,923, which is incorporated herein by reference in its entirety. Certain exemplary methods of producing IESCE have been described in U.S. Pat. No. 11,746,329, which is incorporated herein by reference in its entirety.

In one embodiment, the bioreactor is modified to permit the conditioning chamber to house a cylindrical bio-scaffold sleeve. A substantially higher number of stem cells can be seeded onto the much larger sleeve surface area (about 1 million cells/cm², for example, on the side exposed to flow), and then oscillatory flow-conditioned, to further augment the number of exosomes released into the culture media.

In one embodiment, the bioreactor comprises the pulsatile flow pump that delivers an increased or high oscillatory flow condition compared to a physiological oscillatory flow condition. Such increased or high oscillatory flow condition provides a pathological mechanical environment for cultured stem cells. To specify and quantify the mechanical environment, oscillatory shear index (OSI) is utilized as a parameter to show precise flow oscillation magnitudes. OSI is a measure of flow disturbances that quantifies the ratio between the forward flow net temporal shear stress to the total temporal shear stress magnitude that is assumed to be always positive (Equation (1)), and the OSI value ranges between zero (no oscillation, or steady flow) to 0.5 (full oscillation, or forward flow in half the temporal cycle and reversed flow in the other half).

$$OSI = \frac{1}{2} ( 1 - \frac{\vert {\int }_{0}^{T} {τ}_{ω} dt \vert}{{\int }_{0}^{T} \vert {τ}_{ω} dt \vert} ) \qquad ( 1 )$$

Where T=duration of cycle, t=time, and τ_{ω}=wall shear stress.

The “pathological,” “pathologically high” and/or “high” oscillation flow condition refer to a flow condition having an OSI that produces a mechanical environment for the cultured cells, which coincides with that of cardiovascular disease. The pathological oscillatory flow condition provides abnormal dynamic mechanical environments in the bioreactor that mimics blood flow in a cardiovascular disease state. In specific embodiments, the pathological/high oscillatory flow condition has a shear index/oscillatory shear index (OSI) higher than that of a physiological oscillatory flow condition. Preferably, the pathological/high oscillatory flow condition has a shear index/oscillatory shear index (OSI) ranging from about 0.3 to about 0.5. In a specific embodiment, the pathological/high oscillatory flow condition has a shear index/OSI of about 0.5.

The pulsatile flow pump can deliver oscillatory flow conditions of relevance to the cardiovascular system (a physiological oscillatory shear index/OSI, for example, 0.18≤OSI≤0.23).

Conditioning of stem cells on a bio-scaffold substrate for a period of 2 weeks at a physiologically-relevant oscillatory shear stress and directionality (3-5 dynes/cm2; OSI=0.2) or a pathological/high oscillatory flow condition (OSI=0.5), after an initial 8 day static culture period will lead to the secretion of IESCE into the media. Finally, centrifugation and precipitation-based exosome isolation procedures followed by filtration steps will yield IESCE.

Specific biomechanical fluid-induced stress conditions were identified that permit scaffold-seeded human BMSCs to secrete augmented biological factors into the culture media that promote de novo cardiovascular ECM formation. These factors can be isolated and delivered to damaged heart tissues as an injectable therapeutic.

Analyses of MSC conditioned medium indicates that MSCs secrete mediators of tissue repair, including growth factors, cytokines and chemokines. These factors provide the paracrine signaling and cell-cell interactions that stimulate native parenchymal cells to initiate repair of damaged issues. This signaling regulates an array of regenerative functions such as modulating the local immune system, enhancing angiogenesis, preventing cell apoptosis as well as stimulating survival, proliferation and differentiation of resident tissue-specific cells.

Key cardiac repair factors released by MSCs include VEGF, TGF-β1, FGF-2, HGF and G-CSF. With the secretion of growth factors, extracellular vesicles and exosomes, MSCs act to promote specific biological activities by native progenitor cells that can lead to cardiac tissue repair.

The exosomes can comprise a cocktail of molecular factors that promote cardiac function via neovascularization and de novo tissue formation. Accordingly, certain embodiments of the invention provide IESCE that can be used to treat a heart disease. IESCE accelerates healing and remodeling responses of cardiovascular structures (e.g., heart, blood vessels, and heart valve) damaged from disease.

In one embodiment, the subject invention provides a method of producing secreted enhanced exosomes (e.g., IESCEs) from cells, preferably stem cells, such as MSCs and CSCs, the method comprising culturing the cells in a bioreactor system, wherein the bioreactor system comprises one or more bioreactors and the cells are cultured in the bioreactors separately or sequentially in culture medium; collecting the culture medium from the bioreactor system; and isolating exosomes from the culture medium.

In one embodiment, the enhanced exosomes, or IESCE can be produced by a method comprising seeding cells (e.g., stem cells) onto a bio-scaffold; culturing the cells on the bio-scaffold in a bioreactor that delivers a mechanical oscillatory flow condition to the cells on the bio-scaffold; and collecting the enhanced exosomes or IESCE from the culture medium; and optionally, purifying the enhanced exosomes or IESCEs.

In one embodiment, culturing the cells on the bio-scaffold comprises culturing the cells on the bio-scaffold under rotisserie culture followed by culturing the stem cells in a culture medium in the bioreactor under a mechanical oscillatory flow condition (e.g., a physiological oscillatory flow condition or a pathological/high oscillatory flow condition).

In a preferred embodiment, culturing the cells on the bio-scaffold comprises culturing the cells in a culture medium in the bioreactor under a pathological/high oscillatory flow condition, for example, with an OSI of about 0.5.

The term “bioreactor” refers to a cell culture system that provides nutrients to cells and removes metabolites, as well as furnishes a physio-chemical environment conducive to cell growth, in a closed sterile system. In specific embodiments, the biological processes (e.g., secretion of exosomes) develop under monitored and controlled environmental and operating conditions, for example, pH, temperature, pressure, nutrient supply and waste removal. Examples of bioreactors include, but are not limited to, static bioreactors, stirred flask bioreactors, rotating bioreactors, and direct perfusion bioreactors.

In one embodiment, the bioreactor is a flow perfusion bioreactor system that comprises a pulsatile flow pump that provides controlled flow conditions (static and dynamic, e.g., pulsatile and/or oscillatory flow) to the cultured cells.

In specific embodiments, the bioreactor comprises a separable and insertable cylindrical specimen holder, one or more U-shaped bioreactor chambers that house the cell-seeded scaffolds, wherein the bioreactor chambers are in fluidic communication with culture media flow that is facilitated by a programmable pulsatile pump system. In one embodiment, the scaffolds can be placed in each bioreactor chamber in a parallel configuration to the flow direction and fully immersed in the media solution. In some embodiments, the flow perfusion bioreactor system further comprises other components including tubes, glass media bottles, flow probes, pressure sensors and test software used to obtain the hydrodynamic data during the flow and pressure testing phase.

In one embodiment, the bioreactor may be a rotating bioreactor such as a tubespin bioreactor. Such tube can be placed in a rotisserie for rotisserie culture.

In one embodiment, culturing the cells in the bioreactor system comprises seeding the cells on a bio-scaffold, placing the bio-scaffold in a bioreactor or in a chamber of the bioreactor, and culturing the cells in the culture medium under conditions that augment the number of exosomes released into the culture medium.

In one embodiment, the subject invention provides a method for producing IESCEs, the method comprising seeding stem cells onto a bio-scaffold; subjecting the stem cells on the bio-scaffold to rotisserie culture; placing the bio-scaffold in a bioreactor that delivers a pathological/high oscillatory flow condition to the stem cells on the bio-scaffold; culturing the stem cells in a culture medium in the bioreactor under the pathological/high oscillatory flow condition; and collecting IESCEs from the culture medium.

In a preferred embodiment, the bio-scaffold comprises polymer materials that may be biocompatible and biodegradable. Such polymer materials include, but are not limited to, PSIS, PGA, polylactic acid (PLA), poly(lactic-co-glycolic) acid (PLGA), PLLA, and PEG. In specific embodiments, the bio-scaffold is made of co-polymers selected from, PGA, polylactic acid (PLA), poly(lactic-co-glycolic) acid (PLGA), PLLA, and PEG. Preferably, the bio-scaffold comprises PGA-PLLA.

In some embodiments, cells seeded on the bio-scaffold in a bioreactor are cultured to confluency (e.g., 60%-99% or 75-95% confluency, such as about 75%, 80%, 85%, 90% or 95%) in culture medium before collecting the conditioned medium fractions from the bioreactor for isolating exosomes from the medium.

In one embodiment, the method comprises seeding the cells on the bio-scaffold at a concentration of at least about 1×10⁵ cells/mL, 2×10⁵ cells/mL, 5×10⁵ cells/mL, or 1×10⁶ cells/mL.

In one embodiment, the cells used to produce exosomes can be cultured under culture conditions including, for example, static and dynamic culturing, which may be selected from, for example, any one or combinations below:
1) rotisserie culture for N days, N≥1, preferable, N≥7 or 8;
2) no flow culture for X days, X≥1, preferably, X≥7, more preferably, X≥14; and
3) flow culture for Y days, Y≥1, preferably, Y≥7, more preferably, Y≥14, wherein the flow may be selected from high flow (high OSI), the physiological flow (physiological OSI) and low flow (low OSI); and the flow culture may include, for example, continuous flow, oscillatory flow and pulsatile flow culture.

In one embodiment, the pulsatile and/or oscillatory flow pattern is induced by the combination of a cyclic flexure (e.g., 0.5 Hz, 1 Hz, 1.5 Hz, 2 Hz, and any value in between) and steady flow (flex-flow) that induces shear stress, for example, of 2-10 dynes/cm², 2-9 dynes/cm², 3-9 dynes/cm², 2-8 dynes/cm², 3-8 dynes/cm², 2-7 dynes/cm², 3-7 dynes/cm², 2-6 dynes/cm², 3-6 dynes/cm², 2-5 dynes/cm², 3-5 dynes/cm², 2-4 dynes/cm², or 4-5 dynes/cm².

In one embodiment, the OSI ranges from about 0.05 to about 0.5, from about 0.1 to about 0.5, from about 0.1 to about 0.45, from about 0.1 to about 0.4, from about 0.1 to about 0.38, from about 0.1 to about 0.35, from about 0.1 to about 0.3, from about 0.15 to about 0.35, from about 0.18 to about 0.3, from about 0.2 to about 0.28, or from about 0.2 to about 0.25. In specific embodiments, the OSI is 0.1, 0.11, 0.12, 0.13, 0.14, 0.15, 0.16, 0.17, 0.18, 0.119, 0.2, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.4.

In preferred embodiments, the pathological/high oscillatory flow condition has an OSI ranging from about 0.4 to about 0.5. In a specific embodiment, the pathological oscillatory flow condition has an OSI of about 0.5.

In some embodiments, the conditioned medium fractions from the bioreactor may also be collected any time/day under the culture condition above for isolating exosomes. In specific embodiments, the conditioned media from the cells cultured in medium may be collected every 8-100 hours, every 12-72 hours, every 12-60 hours, every 12-48 hours, or every 24-48 hours.

In one embodiment, isolating exosomes from the culture medium comprises filtration and ultracentrifugation of the pooled fractions to obtain an exosome-containing pellet and resuspending the exosome-containing pellet in a buffer, e.g., PBS or saline. In some embodiments, filtration comprises a step of passing the pooled fractions through a filter, such as a 0.2 μm filter. In a further embodiment, isolating comprises a centrifugation step, for example, prior to filtration to remove any cell debris.

In some embodiments, each medium fraction comprises at least 1×10ⁿ exosomes (n≥6). In one embodiment, each medium fraction may also comprise about 1×10⁹ to about 1×10¹⁵, about 1×10¹⁰ to about 1×10¹⁴, about 1×10¹¹ to about 1×10¹³, or about 1×10¹¹ to about 1×10¹², exosomes. In specific embodiments, at least 1×10¹¹, 5×10¹¹, 1×10¹², 5×10¹², 1×10¹³, 5×10¹³, 1×10¹⁴, 5×10¹⁴, or 1×10¹⁵ exosomes are isolated in the collected media fractions.

In one embodiment, the method may further comprise analyzing isolated exosomes using any method that allows direct or indirect visualization of exosomes and may be in vivo or ex vivo. For example, the analysis may include, but is not limited to, ex vivo microscopic or cytometric detection and visualization of exosomes bound to a solid substrate, flow cytometry, fluorescent imaging, and the like. The exosomes may be analyzed by flow cytometric expression of the exosome surfaces markers, and/or transmission electron microscopy (TEM).

In one embodiment, the subject invention provides agents for cardiac regeneration that are exogenous CSCs. Eight sub-types of CSCs have been identified with varying potential for use as regenerative therapy agents:
1) Cardiosphere-Derived Cells (CDCs); clusters of cardiac progenitor, endothelial and mesenchymal cells which have shown improvement in ventricular ejection fraction and reduction in heart failure status.
2) Sca-1+ CSCs; reside in various regions of the human heart and although they have been shown to be capable of differentiating into cardiomyoblasts, no clinical trials have been conducted with this cell type.
3) Cardiac Mesoangioblasts; shown to exhibit differentiation into cardiomyocytes in vitro, transplanted cells however showed limited proliferation, migration or paracrine activity.
4) Cardiac Side Population cells; possess stem-like properties but their ability to differentiate into cardiomyocitesis under debate and they are depleted with advancing age, they are considered unlikely candidates for clinical studies.
5) Islet-1+ CSCs; cells committed towards the cardiomyocyte lineage with differentiation capability towards all three cardiovascular lineages, their scarcity in the human heart limits their use in cell replacement therapy.
6) Epicardium-Derived Progenitor cells; quiescent epicardial cells shown to undergo epithelial-to-mesenchymal transition post infarct and to migrate into the infarcted myocardium to participate in cardiac repair.
7) Cardiac Colony-Forming-Unit Fibroblasts; exhibit MSC properties with proliferation and differentiation capacities into endothelial and smooth muscle cells, but not into cardiomyocytes, suggesting limited potential for regenerating cardiac muscle.
8) W8B2-CSCs; reside in human atrial appendages and can differentiate into cardiovascular lineages, showed low engraftment but powerful paracrine effects with promising potential for cardiac therapy and regeneration.

In one embodiment, the stem cells are MSCs or CSCs. In a preferred embodiment, MSCs are BMSCs. In some embodiments, CSCs are selected from CDCs, Sca-1+ CSCs, Cardiac Mesoangioblasts, Cardiac Side Population cells, Islet-1+ CSCs, Epicardium-Derived Progenitor cells, Cardiac Colony-Forming-Unit Fibroblasts, and W8B2+ CSCs.

As with MSCs, due to low engraftment of transplanted CSCs, their cardio-preservative benefits can be attributed to the powerful paracrine effects from the release of their exosomes that activate resident stem cells. Preclinical trials show promising results in terms of reduction of infarct size and restoration of cardiac function. CSCs have shown to be superior in terms of in vivo secretion of cardio reparative factors, with W8B2+ and CDC's evidencing greater paracrine potential when compared to BMSCs.

Importantly, CSC-secreted exosomes contain key cardio-protective proteins, lipids and genetic material that can have the cardio-reparative effects of CSCs when injected into the infarcted myocardium. When injected in animal hearts, the cell-free conditioned media containing the released CSC shows cardio-reparative effects equivalent to transplanting the cells, with implications on how an exosome-containing IESCE could be applied to treat ischemic cardiomyopathy.

### Compositions of IESCE

In one embodiment, the subject invention provides compositions for preventing, inhibiting, or reducing scar tissue growth in cardiovascular system. In one embodiment, the subject invention also provides compositions for treating a cardiovascular disease associated with scaring.

In one embodiment, the composition comprises secreted exosomes of stem cells such as bone marrow stem cells and CSCs. Preferably, the BMSCs are bone marrow mesenchymal stem cells (BMSCs). In specific embodiments, the secreted exosomes are exosomes, i.e., IESCE, produced under a pathological oscillatory flow condition.

In one embodiment, the composition comprises IESCE and a pharmaceutically acceptable carrier. In a further embodiment, the composition is cell-free. “Pharmaceutically acceptable carrier” refers to a diluent, adjuvant or excipient with which the one or more active agents disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith.

Examples of carriers suitable for use in the pharmaceutical compositions are known in the art and such embodiments are within the purview of the invention. The pharmaceutically acceptable carriers and excipients, including, but not limited to, aqueous vehicles, water-miscible vehicles, non-aqueous vehicles, stabilizers, solubility enhancers, isotonic agents, buffering agents, suspending and dispersing agents, wetting or emulsifying agents, complexing agents, sequestering or chelating agents, cryoprotectants, lyoprotectants, thickening agents, pH adjusting agents, and inert gases. Other suitable excipients or carriers include, but are not limited to, dextran, glucose, maltose, sorbitol, xylitol, fructose, sucrose, and trehalose.

In one embodiment, the compositions of subject invention or the secreted exosomes of the subject invention, e.g., IESCE, comprise tissue repair factors including VEGF, IL-6, IL-10, TGF-β1, FGF-2, EMMPRIN, MMP-9, HGF and/or G-CSF. In one embodiment, the compositions of subject invention comprises one or more cytokines selected from, for example, TNF-α, IGF-1, VEGF, IL-6, FGF-1, TGF-β, EGF, leptin, IL-1α, PDGF-BB, resistin, MCP-1, and adiponectin.

In certain embodiments, the compositions of the subject invention comprise a significantly increased amount of one or more cytokines compared to that from a control condition and/or a physiological flow condition. The increase in one or more cytokines in the composition according to the subject invention can be at least a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, or any amount of increase in between.

In one embodiment, the compositions of the subject invention or the secreted exosomes comprise one or more cardiac tissue-specific proteins such as serum albumin, serotransferrin, and alpha-2-macroglobulin.

In one embodiment, the compositions of the subjection invention comprise a significantly increased amount of total proteins compared to that from a control condition. The increase in total protein quantity in secreted exosomes according to the subject invention can be about a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, 600%, 700%, 800%, 900%, 1000%, 2000%, 3000%, 4000%, 5000% or more, or any amount of increase in between.

In one embodiment, the composition of the subject invention may further comprise non-exosome components from the culture media, wherein the non-exosome components are secreted from the stem cells in culture. In one embodiment, the non-exosomes components may comprise a total protein of at least 1.5 ng/cell, at least 2 ng/cell, at least 2.5 ng/cell, at least 3 ng/cell, at least 3.5 ng/cell, at least 4 ng/cell, at least 4.5 ng/cell, at least 5 ng/cell, at least 5.5 ng/cell, at least 6 ng/cell, at least 6.5 ng/cell, at least 7 ng/cell, at least 7.5 ng/cell, at least 8 ng/cell, at least 8.5 ng/cell, at least 9 ng/cell, at least 9.5 ng/cell, at least 10 ng/cell, at least 11 ng/cell, at least 12 ng/cell, at least 13 ng/cell, at least 14 ng/cell, at least 15 ng/cell, at least 16 ng/cell, at least 17 ng/cell, at least 18 ng/cell, at least 19 ng/cell, at least 20 ng/cell, at least 21 ng/cell, at least 22 ng/cell, at least 23 ng/cell, at least 24 ng/cell, at least 25 ng/cell, at least 26 ng/cell, at least 27 ng/cell, at least 28 ng/cell, at least 29 ng/cell, at least 30 ng/cell, at least 35 ng/cell, at least 40 ng/cell, at least 45 ng/cell, at least 50 ng/cell, at least 55 ng/cell, at least 60 ng/cell, at least 65 ng/cell, at least 70 ng/cell, at least 75 ng/cell, at least 80 ng/cell, at least 85 ng/cell, at least 90 ng/cell, at least 95 ng/cell, at least 100 ng/cell, or any amount in between.

In one embodiment, the compositions of the subjection invention comprise a significantly increased amount of total proteins from the non-exosome components compared to that from a control condition. The increase in total protein quantity in non-exosome components according to the subject invention can be about a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 110%, 120%, 150%, 200%, 250%, 280%, 300%, 350%, 400%, 450%, 500%, 600%, 700%, 800%, 900%, 1000%, 2000%, 3000%, 4000%, 5000% or more, or any amount of increase in between.

In a specific embodiment, the compositions of the subject invention or the secreted exosomes comprise a significantly reduced amount of fibronectin compared to that from a control condition. The reduction in fibronectin quantity in secreted exosomes and/or the composition according to the subject invention can be about a 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 100%, or any amount of reduction in between.

In one embodiment, the composition is formulated in accordance with routine procedures as a pharmaceutical composition adapted for local administration to, for example, humans. Typically, compositions for local administration are solutions in a sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form. Where the composition is administered by injection, an ampoule of sterile water for injection or saline can be provided so that the ingredients may be mixed prior to administration.

The pharmaceutical compositions may be formulated in any forms that are suitable for parenteral administration, including solutions, suspensions, liposomes, microspheres, and nanosystems suitable for solutions or suspensions in liquid prior to injection.

The compositions of the present invention can be administered to the subject being treated by standard routes, including the local, oral, ophthalmic, nasal, topical, transdermal, intra-articular, parenteral (e.g., intravenous, intraarterial, intraperitoneal, intrathecal, intraventricular, intradermal, intracavity, subcutaneous or intramuscular), intracranial, intracerebral, intraspinal, intrauterine, or rectal route. Depending on the condition being treated, one route may be preferred over others, which can be determined by those skilled in the art. Preferably, the composition is administered by injection (e.g., IV injection), gradual infusion over time or implantation.

Depending on the route of administration, the pharmaceutical composition can be associated with a material to protect the pharmaceutical composition from the action of enzymes, acids, and other natural conditions that may inactivate the pharmaceutical composition. Dispersions can also be prepared in glycerol, liquid polyethylene glycols, and mixtures thereof, and in oils. Under ordinary conditions of storage and use, these preparations may contain a preservative to prevent the growth of microorganisms.

Administration can be carried out using therapeutically effective amounts of the agents described herein for periods of time effective according to the subject invention.

In one embodiment, the pharmaceutical composition comprising secreted exosomes of stem cells according to the invention, together with an adjuvant, carrier, or diluent, may thus be placed into the form of solids including tablets, filled capsules, powder and pellet forms, and liquids such as aqueous or non-aqueous solutions, suspensions, emulsions, elixirs, and capsules filled with the same.

In one embodiment, the composition of the subject invention may comprise a further therapeutic agent. Preferably, the further therapeutic agent comprises one or more cytokines, drugs, nucleic acids, small molecules, or proteins that can be used for treating cardiovascular diseases.

### Therapeutic Uses

Stem cell-injection therapy to the heart has been investigated for the treatment of cardiovascular diseases. However, as stem cell survival in heart tissues is extremely limited, so are their effects.

The trophic effects can, in essence, be replicated by IESCE without the need to inject live cell products that require specialized handling and storage. IESCE is a non-living entity, can be manufactured in scale, is easily stored and avoids the availability and cost issues associated with stem cells such as CSCs. The IESCE can also leverage the beneficial effects of stem cell injections into the damaged heart muscle in a manner that is not cell survival-dependent, thereby enabling further restoration of heart function. In accordance with the subject invention, IESCE can be produced in unlimited quantities to improve cardiac function, thereby overcoming supply limitations of stem cells such as CSCs.

The secretion factors of CSC's have a powerful cardio-protective effect in the ischemic myocardium. Paracrine factors released from transplanted CSCs lead to cardiac repair, thereby contributing towards improved and sustainable cardiac function. The cardio preservative benefits of transplanted CSCs can be attributed to the powerful paracrine effects from the release of their exosomes that activate resident stem cells. Furthermore, the secreted factors presented in the cell conditioning medium have also been shown to improve tissue repair.

Importantly, CSC-secreted exosomes contain key cardio-protective proteins, lipids and genetic material, which can recapitulate the cardio-reparative effects of CSCs when injected into the infarcted myocardium. Of particular note, when injected in animal hearts, the cell-free conditioned media containing the released CSC exosomes has shown cardio-reparative effects equivalent to transplanting the cells, with implications on how an exosome-containing IESCE could be applied to treat ischemic cardiomyopathy.

Administration of cell-free IESCE therapy can provide cardio-reparative effects by activating the paracrine signaling loop in endogenous resident stem cells, which in turn may act as regenerative effectors in situ.

In one embodiment, the subject invention provides a method for treating a cardiovascular disease, the method comprising administering to a subject in need thereof, the composition of the subject invention that comprises exosomes produced according to the method of the subject invention.

In one embodiment, the subject invention provides a method for treating a cardiovascular disease, the method comprising administering to a subject in need thereof, a therapeutically effective amount of exosomes of the subject invention. “Therapeutically effective” refers to the amount of pharmaceutically active compounds/molecules/particles, e.g., exosomes, according to the subject invention that will result in a measurable desired medical or clinical benefit to a patient, as compared to the patient's baseline status or to the status of an untreated or placebo-treated (e.g., not treated with the compound/molecule/particle) subject.

The term “cardiovascular disease” as used herein refers to any disease or disorder affecting the cardiovascular system, including the heart and blood vessels. A vascular disease or disorder includes any disease or disorder characterized by vascular dysfunction. Particularly preferred cardiovascular diseases are selected from the group consisting of atherosclerosis, a coronary heart disease (which involves the blood vessels supplying the heart muscle), a cerebrovascular disease (which involves blood vessels supplying the brain), a peripheral arterial disease (which involves blood vessels supplying the arms and legs), a rheumatic heart disease (leading to damage to the heart muscle and heart valves from rheumatic fever, caused by streptococcal bacteria, and other inflammatory heart conditions), a congenital heart disease (due to malformations of heart structure existing at birth), a deep vein thrombosis and pulmonary embolism from blood clots in the leg veins, an acute coronary symptom (preferably unstable angina pectoris or acute myocardial infarction), stable angina pectoris, stroke (preferably ischemic stroke), ischemic cardiomyopathy, pulmonary hypertension, septic cardiomyopathy, inflammation or autoimmune disease associated arteriosclerosis or restenosis. Other inflammatory heart diseases include inflammation of the heart muscle (myocarditis), the membrane sac (pericarditis) that surround the heart, the inner lining of the heart (endocarditis) or the myocardium (heart muscle).

In certain embodiments, the cardiovascular disease is an inflammatory heart disease such as cardiomyopathy, pericardial disease, and valvular heart disease. In certain embodiments, the cardiovascular diseases are associated with scarring, fibrosis, structural abnormality and/or dysfunction of the heart. In a specific embodiment, the cardiovascular disease associated with scarring is myocardial infarction.

In a specific embodiment, the cardiovascular disease is cardiomyopathy. The term “cardiomyopathy” refers to a myocardial disease accompanied by cardiac dysfunction. Also, cardiomyopathy is often accompanied by structural abnormalities of the heart, such as cardiomegaly, cardiomyocyte hypertrophy, and myocardial fibrosis. One of the most common types of cardiomyopathy is idiopathic dilated cardiomyopathy, where the heart is enlarged.

In certain embodiments, cardiomyopathy is selected from, for example, dilated cardiomyopathy, hypertrophic cardiomyopathy, restrictive cardiomyopathy, ischemic cardiomyopathy, hypertensive cardiomyopathy, valvular cardiomyopathy, drug-induced cardiomyopathy, alcoholic cardiomyopathy, mitochondrial cardiomyopathy, cardiomyopathy caused by cardiac sarcoidosis, cardiomyopathy caused by cardiac amyloidosis, cardiomyopathy caused by myocarditis, cardiomyopathy caused by muscular dystrophy, cardiomyopathy caused by cardiac Fabry's disease, and peripartum cardiomyopathy.

In a specific embodiment, the cardiovascular disease is selected from myocardial infarction, ischemic cardiomyopathy, stroke, pulmonary hypertension, coronary heart disease, acute coronary syndromes (ACS), heart valve disease and septic cardiomyopathy.

The term “subject” or “patient,” as used herein, refers to an organism, including mammals such as primates. Mammalian species that can benefit from the disclosed methods of treatment include, but are not limited to, apes, chimpanzees, orangutans, humans, and monkeys; domesticated animals such as dogs and cats; livestock such as horses, cattle, pigs, sheep, goats, and chickens; and other animals such as mice, rats, guinea pigs, and hamsters. Preferably, the subject is a human.

The terms “treatment” or any grammatical variation thereof (e.g., treat, treating, etc.), as used herein, includes but is not limited to, the application or administration to a subject (or application or administration to a cell or tissue of a subject) with the purpose of delaying, slowing, stabilizing, curing, healing, alleviating, relieving, altering, remedying, less worsening, ameliorating, improving, or affecting a disease or condition, the symptom of the disease or condition, or the risk of (or susceptibility to) the disease or condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical or mental well-being.

The compositions of the present invention can be administered to the subject being treated by standard routes, including the local, oral, ophthalmic, nasal, topical, transdermal, intra-articular, parenteral (e.g., intravenous, intraarterial, intraperitoneal, intrathecal, intraventricular, intradermal, intracavity, subcutaneous or intramuscular), intracranial, intracerebral, intraspinal, intrauterine, or rectal route. Depending on the condition being treated, one route may be preferred over others, which can be determined by those skilled in the art. Preferably, the composition is administered by injection (e.g., IV injection), gradual infusion over time or implantation.

In a preferred embodiment, the composition of the subject invention or IESCE can be injected onto adversely affected regions via intra-cardiac injections. Multiple injection sites, e.g., 2, 3, 4, or 5 injection sites may be selected for such injection.

In certain embodiments, the stem cells such as MSCs and CSCs from which the exosomes are derived can be autologous, allogeneic or xenogeneic. As used herein, the term “autologous” means that the donor of the stem cells such as MSCs and CSCs and the recipient of the exosome (or isolated exosome population) derived from said stem cells such as MSCs and CSCs are the same subject. The term “allogeneic” means that the donor of the stem cells such as MSCs and CSCs and the recipient of the exosome (or isolated exosome population) derived from said stem cells such as MSCs and CSCs are different subjects. The term “xenogeneic” means that the donor of the stem cells such as MSCs and CSCs and the recipient of the exosome (or isolated exosome population) derived from said stem cells such as MSCs and CSCs are subjects of different species. In a particular embodiment, the stem cells such as MSCs and CSCs from which the exosomes derived are allogeneic.

In one embodiment, the subject invention provides a method for promoting tissue regeneration and/or repair, the method comprising identifying a damaged tissue in a subject in need of regeneration and/or repair, and administering to the subject the composition of the subject invention that comprises exosomes produced according to the method of the subject invention.

In one embodiment, the subject invention provides a method for promoting tissue regeneration and/or repair, the method comprising identifying a damaged tissue in a subject in need of regeneration and/or repair, and administering to the subject a therapeutically effective amount of exosomes of the subject invention.

In one embodiment, the tissue in need of regeneration and/or repair includes, for example, damaged tissues, necrotic tissues, tissues after surgery, tissues with reduced function, fibrosing tissues, aged tissues, and diseased tissues. Examples of the tissues include live skin tissues and tissues obtained by internal biopsy (surgery) (brain, lung, heart, liver, stomach, small intestine, large intestine, pancreas, kidney, urinary bladder, spleen, uterus, testis, blood, bone, cartilage, fat, skeletal muscle, epithelial tissues etc.).

The method of the subject invention contributes to functional regeneration of the tissue in need of regeneration and maintenance/enhancement of function. In the present invention, examples of tissue in need of regeneration include, but are not limited to, tissues damaged by various pathological conditions due to ischemic/hypoperfusive/hypoxic conditions, trauma, burns, inflammation, autoimmunity, gene abnormalities, and the like. With use of the above tissue regeneration-promoting agents and compositions, treatments for inducing functional tissue regeneration becomes possible not only in cutaneous diseases such as intractable cutaneous ulcers, skin wounds, bullosis, and alopecia, but also in tissues in need of regeneration such as cerebral infarction, myocardial infarction, bone fracture, pulmonary infarction, gastric ulcers, and enteritis.

In one embodiment, the subject invention provides a method for preventing scar tissue growth in a cardiovascular system, the method comprising administering, to a subject, the composition of the subject invention that comprises exosomes produced according to the method of the subject invention. Preferably, the subject has a cardiovascular disease, or has been diagnosed with a cardiovascular disease.

In one embodiment, the subject invention provides a method for preventing scar tissue growth in a cardiovascular system, the method comprising administering to a subject, a therapeutically effective amount of exosomes of the subject invention. Preferably, the subject has a cardiovascular disease, or has been diagnosed with a cardiovascular disease.

The term “prevention” or any grammatical variation thereof (e.g., prevent, preventing, etc.), as used herein, includes but is not limited to, at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). The term “prevention” may refer to avoiding, delaying, forestalling, or minimizing one or more unwanted features associated with a disease or disorder, and/or completely or almost completely preventing the development of a disease or disorder and its symptoms altogether. Prevention can further include, but does not require, absolute or complete prevention, meaning the disease or disorder may still develop at a later time and/or with a lesser severity than it would without preventative measures.

In one embodiment, the subject invention provides a method for inhibiting, reducing, or slowing down scar tissue growth in a cardiovascular system, the method comprising administering to, a subject in need, the composition of the subject invention that comprises exosomes produced according to the method of the subject invention. Preferably, the subject has a cardiovascular disease, or has been diagnosed with a cardiovascular disease.

In one embodiment, the subject invention provides a method for inhibiting, reducing, or slowing down scar tissue growth in a cardiovascular system, the method comprising administering to, a subject in need, a therapeutically effective amount of exosomes of the subject invention. Preferably, the subject has a cardiovascular disease, or has been diagnosed with a cardiovascular disease.

In one embodiment, the subject invention provides a method for treating scar tissue in a cardiovascular system, the method comprising administering to the subject the composition of the subject invention that comprises exosomes produced according to the method of the subject invention.

In one embodiment, the subject invention provides a method for treating scar tissue in a cardiovascular system, the method comprising administering to the subject a therapeutically effective amount of exosomes of the subject invention.

In one embodiment, the subject invention provides a method for scar tissue healing in a cardiovascular system, the method comprising identifying a damaged cardiovascular tissue in a subject in need of healing, and administering to the subject the composition of the subject invention that comprises exosomes produced according to the method of the subject invention.

In one embodiment, the subject invention provides a method for scar tissue healing in a cardiovascular system, the method comprising identifying a damaged cardiovascular tissue in a subject in need of healing, and administering to the subject a therapeutically effective amount of exosomes of the subject invention.

In a further embodiment, the method of the subject invention further comprises a step of evaluating the scar tissue in cardiovascular system, e.g., scar size, heart function, and blood flow.

In one embodiment, the compositions can be administered to a subject by methods including, but not limited to, (i) administration through oral pathways, which administration includes administration in capsule, tablet, granule, spray, syrup, or other such forms; (ii) administration through non-oral pathways, which administration includes administration as an aqueous suspension, an oily preparation or the like or as a drip, suppository, salve, ointment or the like; administration via injection, subcutaneously, intraperitoneally, intravenously, intramuscularly, intradermally, and the like; as well as (iii) administration topically, or as deemed appropriate by those of skill in the art for bringing the compound into contact with living tissue; and (iv) administration via controlled released formulations, depot formulations, and infusion pump delivery.

For example, for parenteral administration in an aqueous solution, the solution should be suitably buffered and the liquid diluent first rendered isotonic with sufficient saline or glucose. Sterile aqueous media that can be employed will be known to those of skill in the art in light of the present disclosure. One may also use nasal solutions or sprays, aerosols or inhalants in the present disclosure. Nasal solutions may be aqueous solutions designed to be administered to the nasal passages in drops or sprays.

Oral formulations can include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. A person of ordinary skill in the art would be familiar with well-known techniques for preparation of oral formulations.

The subject invention contemplates the use of compositions that are sterile solutions for intravascular injection or for application by any other route. A person of ordinary skill in the art would be familiar with techniques for generating sterile solutions for injection or application by any other route. Sterile injectable solutions are prepared by incorporating the active agents in the required amount in the appropriate solvent with various of the other ingredients familiar to a person of skill in the art.

For the treatment of disease, the appropriate dosage of a therapeutic composition will depend on the type of disease to be treated, as defined above, the severity and course of the disease, the patient's clinical history and response to the agent, and the discretion of the attending physician. The composition of the subject invention is suitably administered to the patient at one time or over a series of treatments.

Administration can be carried out using therapeutically effective amounts of the agents described herein for periods of time effective according to the subject invention. The effective amount may be determined by one of ordinary skill in the art and includes exemplary dosage amounts for a subject of from about 0.005 to about 500 mg/kg of body weight of active compound per day, which may be administered in a single dose or in the form of individual divided doses, such as from 1 to 4 times per day.

Alternatively, the dosage amount may be from about 0.01 to about 400 mg/kg of body weight of active agent per day, from about 0.05 to about 300 mg/kg of body weight of active agent per day, from about 0.1 to about 200 mg/kg of body weight of active agent per day, from about 0.1 to about 150 mg/kg of body weight of active agent per day, from about 0.2 to about 100 mg/kg of body weight of active agent per day, from about 0.5 to about 100 mg/kg of body weight of active agent per day, from about 0.5 to 50 mg/kg of body weight of active agent per day, from about 1 to about 50 mg/kg of body weight of active agent per day, from about 1 to about 25 mg/kg of body weight of active agent per day, or from about 1 to about 10 mg/kg of body weight of active agent per day.

In specific embodiments, the composition of the subject invention may be administered at least once a day, twice a day, or three times a day for consecutive days, e.g. 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. The composition of the subject invention may also be administered for weeks, months or years.

As used herein, the singular forms “a,” “an,” and “the” are intended to include the plural forms as well, unless the context clearly indicates otherwise. Furthermore, to the extent that the terms “including.” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

As used herein, each of n, N, X and Y is intended to include ≥1, ≥2, ≥3, ≥4, ≥5, ≥6, ≥7, ≥8, ≥9, ≥10, ≥11, ≥12, ≥13, ≥14, ≥15, ≥16, ≥17, ≥18, ≥19, ≥20, ≥21, ≥22, ≥23, ≥ 24, ≥25, ≥26, ≥27, ≥28, ≥29, and ≥30.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

It should be understood that the examples and embodiments described herein are for illustrative purposes only and that various modifications or changes in light thereof will be suggested to persons skilled in the art and are to be included within the spirit and purview of this application.

### Examples

### Materials and Methods

### Human Mesenchymal Stem Cells Culture

Human mesenchymal stem cells (MSCs; RoosterBio, Frederick, MD) were cultured in cell culture media (RoosterBasal™-MSC, RoosterBio, Frederick, MD) and supplement (RoosterBooster™-MSC-XF, RoosterBio, Frederick, MD) with 1% penicillin and streptomycin (Thermo Scientific™ HyClone™, Fisher Scientific) until confluency.

### Scaffold Preparation and Cell Seeding

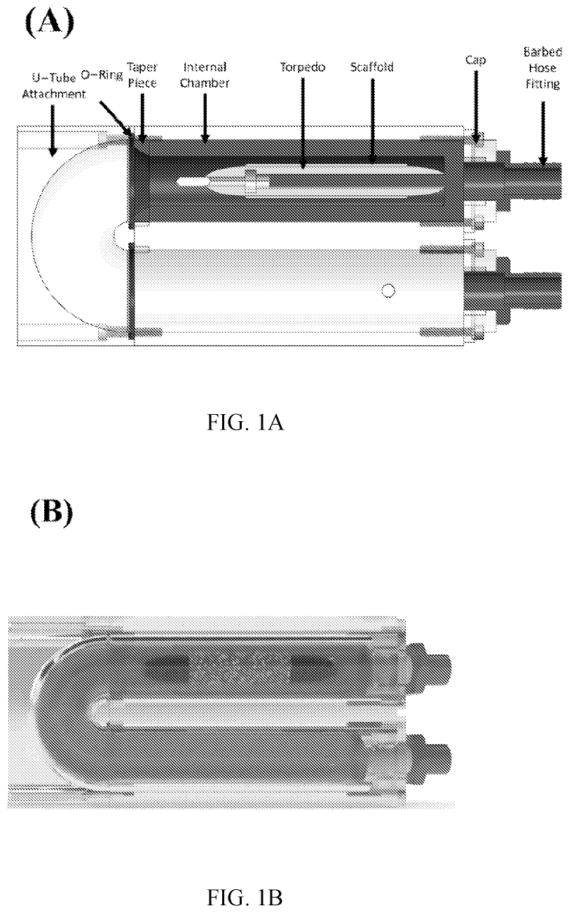

A custom-built, in-house “torpedo” bioreactor (FIGS. 1A and 1B) was utilized to apply oscillatory flow of culture media onto porcine small intestine submucosa (PSIS; Cormatrix Inc., Roswell, GA) bio-scaffolds. The magnitude of the flow oscillations was quantified via the oscillatory shear index (OSI) metric as being between 0≤OSI≤0.5 with 0 being unidirectional (steady) flow and 0.5 being flow with maximum oscillations.

The PSIS bio-scaffold valves were placed on a torpedo holder to secure the bio-scaffolds. The bio-scaffolds were seeded with MSCs in rotisserie culture for 8 days (7 million cells per PSIS valve). Tissue culture medium used for culture were composed of Dulbecco's modified Eagle's medium (DMEM; Corning, Fisher Scientific, Pittsburgh, PA), 10% fetal bovine serum (Corning, Fisher Scientific, Pittsburgh, PA), 1% penicillin and streptomycin, 2 ng/ml basic fibroblast growth factor (bFGF; Corning™, Fisher Scientific) and 82 μg/ml ascorbic acid 2 phosphate (AA2P; Sigma-Aldrich, Dorset, UK). After 8 days of rotisserie culture, the first group continued another 14 days of rotisserie (static), the second group proceeded to the torpedo bioreactor conditioning environment (dynamic) for 14 days under physiological oscillatory flow, and the third group proceeded to the torpedo bioreactor conditioning environment (dynamic) for 14 days under pathological oscillatory flow.

The bioreactors were then connected to a pneumatic piston pulsatile flow pump, with a pump head module (ViVitro Labs Inc., Victoria, BC, Canada) that housed the tubing to transport media to the specimens housed within the bioreactors. Physiologically relevant fluid-induced wall shear stresses (WSS) of 3-9 dynes/cm2 can be conducive to proper cell conditioning. Total culture time of the engineered tissue constructs in both the static and bioreactor time was therefore 22 days, which consisted of 8 days rotisserie culture followed by either an additional 14 days static or 14 days pulsatile flow culture.

At the end of day 22, tissue culture medium from both static and dynamic groups were collected. Medium was centrifuged at 3000×g for 15 minutes. Supernatant was transferred to a sterile vessel and adequate amount of ExoQuick-TC precipitation solution (System Biosciences, Palo Alto, CA). The mixture was stored overnight at 4° C. Afterwards, the mixture was centrifuged at 1500×g for 30 minutes. Supernatant was saved for non-exosomal analysis. Exosomes pellet were resuspended and collected.

### Example 1—BMSC-Seeded Bioscaffolds

BMSC-seeded bioscaffolds subjected to static, physiologically-relevant pulsatile flow, and pathological/high oscillatory flow in vitro lead to accumulation of a potent BMSC-derived exosome product in the conditioned media, i.e., the IESCEs, which contain a rich presence of a trophic factor cocktail that promote cardiac repair and function post-MI, and prevent and slow scar tissue growth in a cardiovascular system. This is because when BMSCS are exposed to fluid-induced oscillatory shear stresses arising from pulsatility, stem cells express cardiovascular-relevant genes and release trophic molecular factors into the media, which aids in regeneration and healing.

Specific components of the bioreactor system include a separable and insertable cylindrical specimen holder, U-shaped bioreactor chambers that housed the hBMMSC-seeded scaffolds, and flow that was facilitated by a programmable pulsatile pump system (ViVitro Labs Inc., Victoria, BC, Canada). Scaffolds can be placed in the bioreactor chamber configured to the flow direction and fully immersed in the media solution. Other components include tubes, glass media bottles, flow probes, pressure sensors and test software used to obtain the hydrodynamic data during the flow and pressure testing phase.

The bioreactor permits the conditioning chamber to house a bio-scaffold sleeve with larger surface, which can be used to seed a substantially higher number of stem cells (about 1 million cells/cm² on the side exposed to flow). To evaluate the IESCE efficacy, three treatments are investigated-(i) exosomes derived from statically cultured, adult, human BMSCs (ii) IESCE derived from physiologically-relevant, oscillatory flow-conditioned adult, human BMSCs and (iii) IESCE derived from pathological oscillatory flow-conditioned adult, human BMSCs.

### Example 2—Assessment of IESCE

Isolated exosomes that form the primary basis for the IESCE, which were taken from BMSCs conditioned in the bioreactor, were assessed with respect to the levels of various cytokines. Eight angiogenic cytokines within the BMSC-secreted exosomes were evaluated where stem cells were cultured under the following conditions: 1) 22 Day Static: 8-day rotisserie and 14-day no flow; 2) 22 Day Physiological: 8-day rotisserie and 14-day bioreactor under physiological flow oscillation (OSI=0.2); and 3) 22 Day Pathological: 8-Day Rotisserie and 14-Day bioreactor under pathological flow oscillation (OSI=0.5).

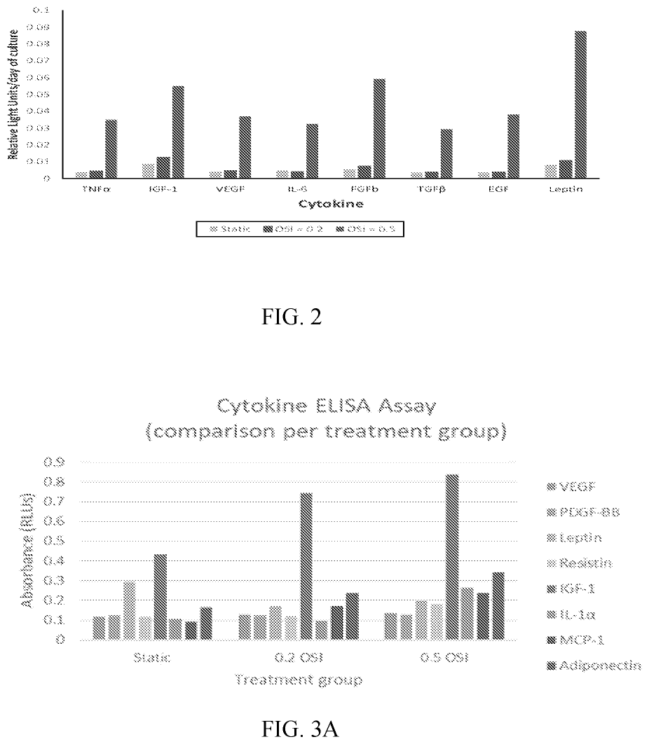

The result shows that a substantial increase in the concentration of all 8 cytokines (Table 1) occurred in the isolated exosomes after BMSCs were exposed to a pathological OSI of 0.5, in comparison to a physiologically relevant OSI flow culture of 0.2 and to static, no flow control culture of BMSCs (FIG. 2). These cytokines are associated with angiogenesis and thus, these exosomes can be used as a nonliving biologic to enhance regeneration of new arteries in the heart to better circumvent cardiac ischemic diseases.

TABLE 1
List of 8 key angiogenic cytokines assessed in exosomes that were isolated
from the conditioned media after BMSCs exposure to high oscillatory
flow conditions (OSI = 0.5) cultured in the torpedo bioreactor.

| Angiogenic Cytokine | Specific Role | Literature Source |
|---|---|---|
| Tumor necrosis factor | Provides protection against | Hadebe et al. |
| alpha (TNF-α) | ischemic-based | |
| | reperfusion injury | |
| Insulin-like growth | Prevents Cardiomyocyte | Sadat et al. |
| factor-1 (IGF-1) | apoptosis | Higashi et al. |
| Vascular | Microvascular and | Schaper, W. and I. |
| endothelial growth | capillary growth | Buschmann. |
| factor (VEGF) | | |
| Interleukin-6 (IL-6) | A dominant | Kanda, T. and T. |
| | angiogenesis-inducing | Takahashi |
| | factor | |
| Acidic fibroblast | Endothelial cell | Slavin, J., |
| growth factor | proliferation | |
| (FGF-1) | | |
| Transforming | Angiogenesis and collagen | Bujak, M. and N.G. |
| growth factor | extracellular matrix | Frangogiannis, |
| type beta (TGF-β) | production | Roberts et al. |
| Epidermal | A dominant | Makki et al. |
| Growth Factor | angiogenesis-inducing | |
| (EGF) | factor with | |
| | co-facilitation of | |
| | VEGF expression | |
| Leptin | Antiatherogenic properties | Koh et al. |

Additional cytokines were also evaluated within the secreted exosomes from cultured stem cells at high oscillatory flow. These exosomes are enhanced, and therefore have the potential to stop or reduce scar tissue growth in the cardiovascular system.

Further, cytokines released from isolated exosomes were analyzed for both groups, i.e., static (n=3) and bioreactor (n=3), using a commercially available ELISA assay (Signosis, Inc., Santa Clara, CA). The following 8 cytokines of relevance to cardiac function were assessed: vascular endothelial growth factor (VEGF), resistin, leptin, insulin-like growth factor-1 (IGF-1), interleukin-1α (IL-1α), monocyte chemoattractant protein-1 (MCP-1), platelet-derived growth factor (PDGF) and adiponectin. VEGF, leptin, IGF-1, IL-1α, MCP-1 adiponectin, and PDGF were selected as anti-apoptotic cardioprotective markers, while resistin was selected as a pro-inflammatory marker.

The assessment was completed at room temperature by following the manufacturer's protocol. Exosomes were first lysed with cell lysate buffer (Signosis, Inc.). Lysed exosome samples were added to the plate and incubated for 2 hours with gentle shaking. After three washes with washing buffer, a biotin-labeled antibody mixture was added to the samples and incubated for 1 hour. Next, a streptavidin-horseradish peroxidase (HRP) solution was added and incubated for 30 minutes. Subsequently, the prepared substrate solution was added to the samples and incubated for 2 minutes. The final samples were read by a spectrophotometer (Synergy HT, BioTek Instruments Inc., Winooski, VT) and reported as relative light units (RLUs). RLUs were then converted to protein concentrations (μg/mL) using the standards curve of the BCA assay obtained for the western blotting.

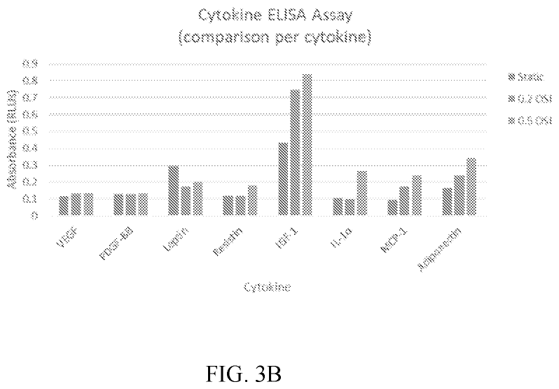

The results from ELISA cytokine panel colorimetric assay show the levels of cytokines such as PDGF-BB, leptin, resistin, IGF-1, IL-1α, MCP-1, and adiponectin (Table 2) in the isolated exosomes after BMSCs were exposed to a pathological OSI of 0.5, in comparison to a physiologically relevant OSI flow culture of 0.2 and to static control culture of BMSCs (FIGS. 3A and 3B).

TABLE 2
cytokines assessed in exosomes that were isolated from the
conditioned media after BMSCs exposure to static control
(OSI = 0), physiological (OSI = 0.2), and high
oscillatory flow conditions (OSI = 0.5).

| | Static | 0.2 OSI | 0.5 OSI |
|---|---|---|---|
| | 0.1165 | 0.13 | 0.136 |
| PDGF-BB | 0.1255 | 0.125 | 0.13 |
| Leptin | 0.2935 | 0.172 | 0.199 |
| Resistin | 0.1185 | 0.12 | 0.178 |
| IGF-1 | 0.4335 | 0.744 | 0.837 |
| IL-1α | 0.106 | 0.096 | 0.266 |
| MCP-1 | 0.092 | 0.172 | 0.238 |
| Adiponectin | 0.165 | 0.24 | 0.342 |

For example, the maximum amounts of the following cytokines were found after the 0.5 OSI culture of the stem cells in the exosomes: IGF-1, MCP-1, and Adiponectin. IGF-1 helps benefit cardiac development. MCP-1 promotes cardiac repair and protection. Adiponectin can prevent pathological remodeling in the heart. Thus, with these 3 cytokines being maximized in the exosomes derived from the 0.5 OSI stem cell culture, cardiac scar tissue formation will reduce after treatment of the cardiovascular tissues with the 0.5 OSI derived exosomes.

In the case of a heart attack, a patient's heart is eventually converted to scar tissue. The enhanced exosomes under high oscillatory flow culture of stem cells (e.g., exosomes obtained from 0.5 OSI culture of the stem cells) can ensure that scar tissue growth can be defeated in the cardiovascular system with the enhanced exosome treatment. Also, it's important that exosomes are not living biologic agents and thus, they would not be subjected to any hostile immune response.

In summary, enhanced exosomal cargo can be secreted by BMSCs by mechanical conditioning at high oscillatory flow conditions, a pathological environment that promotes high quality protein cargo within their secreted exosomes.

The specific mechanical and chemical environments in the bioreactor of the subject invention can be varied to yield even higher concentrations of cardiac therapeutic proteins particularly when these conditions mimic pathology. For example, hypoxic conditions increase the number of exosomes secreted by stem cells and these exosomes contain molecular cargo of higher quality. Hypoxic conditions can be combined with abnormal dynamic mechanical environments in the bioreactor that mimics blood flow in a cardiovascular disease state. Both biomechanical and biochemical conditions (OSI=0.5+hypoxia) could be combined to facilitate a synergistic improvement in the BMSC-secreted exosomal cargo for the purposes of maximizing cardiac regeneration and repair following adverse disease effects on heart tissues.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting.

1. An injectable enhanced stem cell exosome (IESCE) secreted from stem cells under a pathological oscillatory flow condition.

2. The IESCE of claim 1, comprising one or more cytokines selected tumor necrosis factor alpha (TNF-α), insulin-like growth factor-1 (IGF-1), vascular endothelial growth factor (VEGF), interleukin-6 (IL-6), acidic fibroblast growth factor (FGF-1), transforming growth factor type beta (TGF-β), epidermal Growth Factor (EGF), leptin, interleukin-α (IL-1α), Platelet-derived growth factor (PDGF)-BB, resistin, monocyte chemoattractant protein-1 (MCP-1), and adiponectin.

3. The IESCE of claim 1, comprising one or more cardiac tissue-specific proteins.

4. The IESCE of claim 3, the one or more cardiac tissue-specific proteins being selected from serum albumin, serotransferrin, and alpha-2-macroglobulin.

5. The IESCE of claim 1, the stem cells being mesenchymal stem cells (MSCs) or cardiac stem cells (CSCs).

6. The IESCE of claim 1, the MSCs being bone marrow mesenchymal stem cells (BMSCs).

7. The IESCE of claim 1, the CSCs being cardiosphere-derived cells (CDCs), Sca-1+ CSCs, Cardiac Mesoangioblasts, Cardiac Side Population cells, Islet-1+ CSCs, Epicardium-Derived Progenitor cells, Cardiac Colony-Forming-Unit Fibroblasts, or W8B2+ CSCs.

8. The IESCE of claim 1, the pathological oscillatory flow condition having an OSI of about 0.5.

9. A composition comprising the IESCE of claim 1.

10. A method for producing injectable enhanced stem cell exosomes (IESCEs), the method comprising
seeding mesenchymal stem cells (MSCs) or cardiac stem cells (CSCs) onto a polymeric bio-scaffold;
culturing the MSCs or CSCs in a culture medium for the MSCs or CSCs on the polymeric bio-scaffold under rotisserie culture;
placing the polymeric bio-scaffold in a torpedo bioreactor and culturing the MSCs or CSCs under a pathological oscillatory flow condition having a shear index/oscillatory shear index (OSI) of about 0.5;
collecting IESCEs from the culture medium; and
detecting a level of one or more cytokines selected from tumor necrosis factor alpha (TNF-α), insulin-like growth factor-1 (IGF-1), acidic fibroblast growth factor (FGF-1), epidermal Growth Factor (EGF), and leptin in IESCEs, IESCEs comprising an increased level of said one or more cytokines as compared to those of static culture and/or those under a physiological oscillatory flow condition.

11. (canceled)

12. The method of claim 10, the CSCs being cardiosphere-derived cells (CDCs), Stem cell antigen 1 (Sca-1)+ CSCs, Cardiac Mesoangioblasts, Cardiac Side Population cells, Islet-1+ CSCs, Epicardium-Derived Progenitor cells, Cardiac Colony-Forming-Unit Fibroblasts, or mesenchymal stem cell antigen-1 (W8B2)+ CSCs.

13. The method of claim 10, the MSCs being bone marrow mesenchymal stem cells (BMSCs).

14. The method of claim 10, the torpedo bioreactor comprising a pulsatile flow pump that delivers the pathological oscillatory flow condition to the stem cells.

15. (canceled)

16. The method of claim 10, the polymeric bio-scaffold comprising porcine small intestinal submucosa (PSIS), or poly(glycolic acid) (PGA)-poly-1-lactic acid (PLLA).

17. The method of claim 10, further comprising detecting a level of one or more cytokines selected from vascular endothelial growth factor (VEGF), interleukin-6 (IL-6), transforming growth factor type beta (TGF-β), interleukin-α (IL-1α), Platelet-derived growth factor (PDGF)-BB, resistin, monocyte chemoattractant protein-1 (MCP-1), and adiponectin.

18. A method of preventing or reducing scar tissue growth in cardiovascular system, the method comprising administering, to a subject having a cardiovascular disease, the composition of claim 9.

19. The method of claim 18, the cardiovascular disease being a myocardial infarction or cardiomyopathy.

20. The method of claim 15, the administration being via a local, oral, nasal, topical, transdermal, intravenous, intraarterial, intra-cardiac, intraventricular, intradermal, subcutaneous or intramuscular route.

21. The method of claim 10, the MSCs being cultured in the culture medium under rotisserie culture for ≥8 days and in the torpedo bioreactor under the pathological oscillatory flow condition for ≥14 days.

22. The method of claim 10, comprising detecting levels of TNF-α, IGF-1, FGF-1, EGF, and leptin in IESCEs, the IESCEs comprising increased levels of TNF-α, IGF-1, FGF-1, EGF, and leptin as compared to those of static culture and those under a physiological oscillatory flow condition having an OSI of about 0.2.
